(19) United States
(12) Patent Application Publication
(10) Pub. No.: US 2013/0243033 A1
(43) Pub. Date: Sep. 19, 2013

(54) Predicting The Remaining Life Of A Transformer

(71) Applicant: Roberto Asano, JR., Sao Paulo (BR)

(72) Inventor: Roberto Asano, JR., Sao Paulo (BR)

(21) Appl. No.: 13/886,669

(22) Filed: May 3, 2013

Related U.S. Application Data
(63) Continuation of application No. PCT/EP2011/069213, filed on Nov. 2, 2011.

(30) Foreign Application Priority Data
Nov. 3, 2010 (EP) 10189776.7

(51) Int. Cl. G01K 13/00; H01F 27/00

(52) U.S. Cl. CPC G01K 13/00; H01F 27/008
USPC 374/152; 336/58

(57) ABSTRACT

Method of assessing the remaining lifetime of a transformer operating at temperatures above IEC 60° C./65° C./78° C. or IEEE 65° C./65° C./80° C. including a core, a winding conductor covered by high temperature insulation material and pressboard material arranged as an insulation barrier and/or a supporting structure. The covered winding conductor and the pressboard material are immersed in a fluid having a fire point of at least 300° C. The method includes at least twice measuring the temperature of the fluid at its top surface and registering the time of each measurement and assessing the remaining lifetime of the transformer as a function of the measured temperatures and the corresponding registered times, and/or obtaining a sample of pressboard material that has been in contact with the fluid at its top surface and analysing the sample to assess the remaining lifetime of the transformer.

### FIELD OF THE INVENTION

The present invention relates to the field of predicting the age or the remaining life of a transformer comprising at least one winding conductor having high temperature insulation that is immersed in a suitable fluid, wherein pressboard material is employed as an insulation barrier and/or mechanical support spacers.

### BACKGROUND OF THE INVENTION

Transformers normally comprise an oil-immersed core surrounded by winding conductors.

In traditional transformers, the winding conductors were covered by paper. The remaining life of such transformers was often predicted by monitoring the aging of the covering paper.

However, many more modern transformers employ winding conductors covered by high temperature insulation materials having a life expectancy of over 200 years (when predicted in the same way). Consequently, the properties of the cover of the winding conductors are no longer a useful indicative of the remaining life of the transformers. The oil, which normally is mineral oil, is the weakest point of such transformers, limiting the temperature of operation and reducing the importance of life prediction.

In a new generation of transformers, the high temperature insulation cover of the winding conductors is employed in combination with a high fire point fluid, and paper board or cellulose material is mainly used as insulation barriers and/or supporting structures (e.g. spacers).

WO 01/09906 discloses performing temperature measurements on the fluid (typically oil) of a fluid filled transformer for detecting the health of the transformer, specifically whether the transformer has failed or will fail in the near future. It is stated in WO 01/09906 that the fluid or oil shall not be monitored adjacent to a tank wall or near the top level of the fluid in the tank as the fluid in these regions tends to be more stagnant compared to fluid in other regions of the tank. Instead, WO 01/09906 strongly advocates making measurements where the fluid is streaming. Further, WO 01/09906 is silent about operating temperatures.

US 2007/289367 discloses a method of assessing the water content of solid insulation in transformers based i.a. on temperature measurements of the oil in a top portion of the tank. The winding conductors of US 2007/289367 are however not covered by high temperature insulation materials. Instead, US 2007/289367 only mentions paper as an insulation material. US 2007/289367 is also silent about operating temperatures.

DE 10 2007 026175 discloses the calculation of an aging rate of a transformer and one of the following parameters is measured: oxygen content, moisture content, neutralization degree, acidity, soaping degree or over voltage. Further, DE 10 2007 026175 discusses the measurement of the “hot spot” temperature, which is the highest temperature in the insulation material covering the windings. As the hot spot temperature is not the determinant for the aging rate in transformers using high temperature insulation materials, the disclosure of DE 10 2007 026175 is not applicable to such transformers.

WO 98/44356 discloses a device for detecting partial discharges in an electrical apparatus placed in a tank filled with oil, wherein the tank wall is provided with at least one drain tap, characterized in that the device comprises a probe which is mounted on a carrier which is movable through the opened drain tap into the tank. The quality of the insulation material in a transformer may thus be assessed. The document relates to how to get a probe into the transformer but is not related to what measurements to make with the probe. As quality of the insulation material is not the determinant for the aging rate in transformers using high temperature insulation materials, the disclosure of WO 98/44356 is not applicable to such transformers.

U.S. Pat. No. 5,773,709 discloses a method and device for sampling an insulating dielectric fluid (e.g. oil) of e.g. a transformer by using heat convection to transport the fluid from the transformer to a measurement unit. The purpose of the measurement may be to indicate failure of the insulating fluid. The quality of the solid structures of a transformer is however not analyzed.

EP 2244089 discloses assessing the water content of the cellulose material of a transformer by on-line measurements of the humidity and the temperature of the oil. According to EP2244089, the dielectric strength of an arbitrary transformer oil always varies with its relative humidity and heavily depends on two variables: the water content in the oil and the temperature of the oil. The water content of the oil is indicative of the water content in the cellulose insulation, which is considered a quality indicator. As quality of the insulation material is not the determinant for the aging rate in transformers using high temperature insulation materials, the disclosure of WO 98/44356 is not applicable to such transformers.

U.S. Pat. No. 4,654,806 discloses a method of continuously monitoring parameters of a transformer in order to observe long-term changes in the operating conditions, including the insulating oil. Solid insulation appears not to be discussed. The disclosure of U.S. Pat. No. 4,654,806 relate to older transformers in which high temperature insulation materials are not used.

### SUMMARY OF THE INVENTION

It is an object of the present disclosure to provide for the assessment of the age or the prediction of the remaining life of a transformer comprising a winding conductor covered by high temperature insulation material and a pressboard material arranged as a insulation barrier or supporting structure, wherein said covered winding conductor and said pressboard material are immersed in a suitable transformer fluid heated to a temperature higher than conventional.

Thus, there is provided a method of assessing the remaining lifetime of a transformer comprising a core, a winding conductor covered by high temperature insulation material and pressboard material arranged as a insulation barrier and/or a supporting structure, wherein said covered winding conductor and said pressboard material are immersed in a fluid, said method comprising the steps of:
a) at least once measuring the temperature of the fluid at its top surface and registering the time of each measurement; and
b) assessing the remaining lifetime of the transformer as a function of the measured temperature(s) and the corresponding registered time(s),
and/or
a′) obtaining a sample of pressboard material that has been in contact with the fluid at its top surface; and
b′) analysing the sample to assess the remaining lifetime of the transformer.

Further, there is provided a transformer comprising a tank filled with a fluid in which a core, a winding conductor covered by high temperature insulation material and pressboard material arranged as a insulation barrier and/or a supporting structure are immersed, wherein a temperature sensor and/or a fluid-permeable case for holding a piece of pressboard accessible from the exterior of the transformer is/are immersed in the fluid at its top surface.

### BRIEF DESCRIPTION OF THE DRAWINGS

FIG. 1 is a simplified view of a transformer according to one embodiment of the invention.

FIG. 2 shows an example of a sampling model, wherein the x-axis represents time, the y-axis represents an aging variable and the dotted line represents an end-of-life threshold.

FIG. 3 shows an example of a temperature model, wherein the x-axis represents time, the y-axis represents accumulated temperature-dependent life consumption (∫life consumption (temperature, time) dtime) and the dotted line represents expected life.

FIG. 4 is a simplified view of a part of a transformer, showing the core and the windings, as well as insulating, spacing and supporting structures.

### DETAILED DESCRIPTION OF THE INVENTION

The inventive concept of the present disclosure is normally implemented in equipment designed to operate at temperatures above IEC 60° C./65° C./78° C. (top-liquid temperature rise/average winding temperature rise/hot-spot winding temperature rise) or IEEE 65° C./65° C./80° C. (liquid temperature rise/average winding temperature rise/maximum (hottest-spot) winding temperature rise), which use high temperature insulation material for the windings and cellulose or pressboard material for the insulation barrier and/or a supporting structures.

The person skilled in the art of transformers is familiar with temperature terms above. Further, the definition of the terms can be found in standards IEC 60076-2 and IEEE C.57.12.00, respectively.

The term high temperature insulation material is discussed below. The invention is based on the inventor's insight that the weakest point, in determining the lifetime, of the new generation of transformers is neither the cover of the conducting windings nor the fluid, but the cellulose or pressboard components in contact with the fluid of the highest temperature. Further, the fluid of the highest temperature has the lowest density and is therefore found at the top surface.

Accordingly, one or more properties of a cellulose or pressboard sample that has been in contact with the hottest fluid may be measured to predict the remaining lifetime of the transformer. The sample may be obtained from supporting structures or insulation barrier components. Alternatively, the sample may be obtained from a piece of pressboard or cellulose in contact with the hottest fluid, which piece was arranged in the transformer mainly for the provision of such samples. As an example, the sample may be obtained from a position within 50 cm of the top surface.

The prediction may for example comprise a comparison between the sample that has been in contact with the hottest fluid and a sample of fresh pressboard.

For example, a variable that represents the aging of the sample (an “aging variable”) may be measured using a physical, electrical and/or chemical test known to the skilled person following adequate standardized procedures. The aging variable may thus be a quantifiable visual parameter (“appearance”). It may also be a degree of polymerization, tensile strength and/or compression, flexion strength or other mechanical, physical or chemical test that provides a comparison parameter related to the degradation of the structure or its by-products. Further, it may be an electrical property such as resistivity, conductivity, capacitance, dielectric breakdown, power factor, dielectric frequency response, polarization or depolarization current measurement.

However, it is also possible to make the prediction based on measurements of the temperature of the fluid at the top surface. This may be considered an indirect measurement—the higher the temperature of the fluid in contact with the pressboard or cellulose, the higher the deterioration rate of the pressboard or cellulose. Consequently, temperature measurements of the fluid at the top surface can be used in a calculation model to assess the remaining life. The calculation model may be adapted to/calibrated with aging curves of pressboard or cellulose. The aging curves may be empirical data obtained from analyses of pressboard or cellulose material that has been immersed in fluid of various temperatures and different time period.

Further, the direct and the indirect approach may be combined to increase the reliability of the predictions. For example, if the “online” measurements (time and temperature) and the result of the analysis of the test samples (showing the actual ageing) are combined, the prediction model can be calibrated. For example, such a calibration can involve, after a certain time period, a comparison of the remaining life time predicted with the indirect approach with the remaining life time predicted with the indirect approach. The calculation model may then be adjusted based on the result of the comparison. In this manner, the online predictions can be improved over time.

Thus, as a first aspect of the present disclosure, there is provided a method of assessing the remaining lifetime of a transformer operating at temperatures above IEC 60° C./65° C./78° C. or IEEE 65° C./65° C./80° C. comprising a core, a winding conductor covered by high temperature insulation material and pressboard material arranged as a insulation barrier and/or a supporting structure, wherein said covered winding conductor and said pressboard material are immersed in a fluid, said method comprising the steps of:
a) at least once measuring the temperature of the fluid at its top surface and registering the time of each measurement; and
b) assessing the remaining lifetime of the transformer as a function of the measured temperature(s) and the corresponding registered time(s),
and/or
a′) obtaining a sample of pressboard material that has been in contact with the fluid at its top surface; and
b′) analysing the sample to assess the remaining lifetime of the transformer.

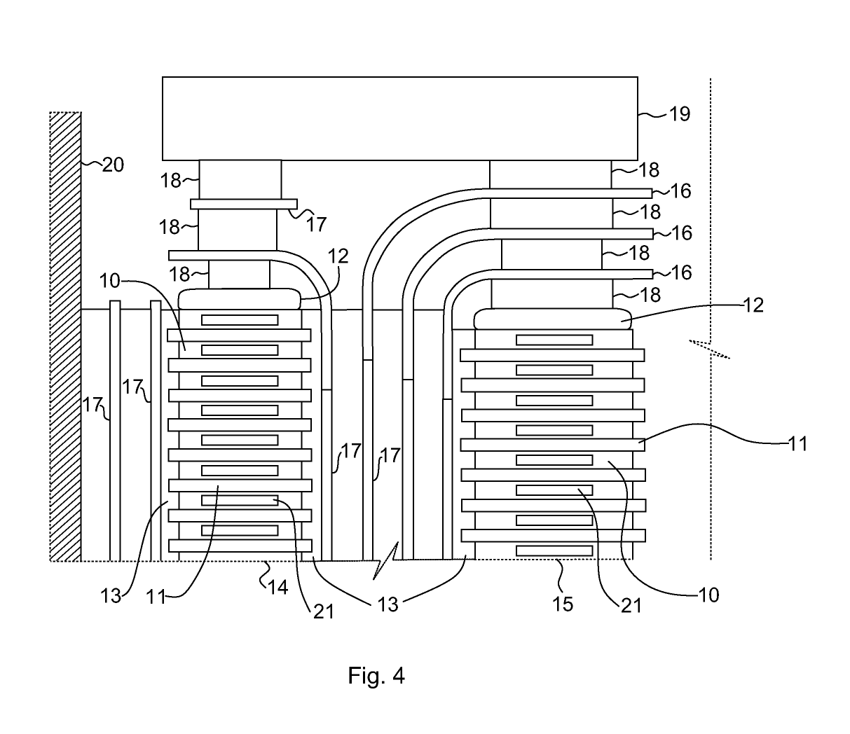

The transformer of the first aspect may comprise more than one core surrounded by winding conductor. In the art, transformers having two or three cores are common. Each core may be surrounded by one, in the case of a reactor, or more winding conductors. For example, one high-voltage winding and one low-voltage winding may surround one core (see also FIG. 4).

The high temperature insulation material of the present disclosure is normally solid. The person of skill in the art of transformers is familiar with the term “high temperature insulation material” and capable of selecting an appropriate type of such a material for a given transformer. For example, the high temperature insulation material of the present disclosure may be a material as defined in IEC 60076-14:2009 or IEEE 1276:1997.

As mentioned above, the highest fluid temperature is found at the top surface of the fluid. In the art, this temperature is sometimes referred to as the “top oil temperature” (see e.g. International Standard IEC 60076-2:1993(E)). The person of skill in the art knows how to measure the top oil temperature (see e.g. IEC 60076-2:1993(E), item 5.3.1).

The fluid of the present disclosure is an insulating fluid suitable for use as a dielectric and cooling medium in transformers. The person of skill in the art is capable of selecting an appropriate fluid for carrying out the invention.

The fluid may for example be a high fire point fluid. The person of skill in the art is familiar with the term “high fire point fluid” in the context of transformers. Normally, such a fluid has a fire-point of at least 300° C. Such fluids are defined in IEC 60076-2 and IEEE C57.12.00.

The fluid may be a mineral oil, but it is not preferred. A mineral oil used in accordance with the present disclosure may have to be exchanged if it ages too much. More preferred is to use an ester-based fluid or a silicone fluid. The ester may be synthetic or natural. An example of a natural ester fluid is biodegradable fluids, such as BIOTEMP™). The high fire point fluid may for example contain at least 50 wt. %, such as at least 75 or 90 wt. % esters.

The pressboard of the present disclosure may for example be paper or cellulose pressed together to form a board. Normally, a board is up to 6 mm thick, and if thicker structures are needed, two or more boards are glued together.

In an embodiment of the method, the average top oil temperature is at least 55° C., such as at least 60° C. or 65° C. In an alternative of complementary embodiment of the method, the average winding temperature is at least 60° C., such as at least 65° C. In yet another alternative of complementary embodiment of the method, the hot spot temperature is at least 75° C., such as at least 80° C. or 85° C.

In step a), the temperature is measured. The time of the measurement is also registered. Normally, this is the time passed since the transformer was installed or since the operation of the transformer was initiated. Provided with the teachings of the present disclosure, the skilled person is capable of selecting an appropriate starting point for the time measurements. When the method of the first aspect is put into practice, many temperature measurements (and corresponding time registrations) will normally be performed, and as the number of measurements increases, the accuracy of the assessment of the remaining life time will normally improve. Thus, in embodiments of the first aspect, the assessment is based on at least 2, such as at least 5, 10, 50, 100 or 500 temperature measurements according to step a). In other words, the temperature measurement of step a) may be performed at least twice, such as at least 5, 10, 50, 100 or 500 times.

In embodiments of the first aspect, the function of b) may be based on at least one empirically determined aging curve of the pressboard material. This is discussed further below under Exemplary embodiments.

The inventor has found that the deterioration of the pressboard material in the transformer of the present disclosure also depends on the level of oxygen and moisture in the transformer fluid. Thus, the accuracy of the prediction may be increased by monitoring the level of these contaminants. Accordingly, step a) may further comprise measuring the level of oxygen and/or water in the fluid in some embodiments of the first aspect. In such embodiments, step b) further comprises assessing the remaining lifetime of the transformer as a function of the measured temperature(s), level(s) of oxygen and/or water and the corresponding registered time(s). However, it is no requirement that the level of oxygen and/or water is measured each time the temperature is measured. For example, the oxygen and/or water measurements may be offline measurements carried out less frequently than the (online) temperature measurements.

In an embodiment of the first aspect, the sample of a′) may be obtained from the insulation barrier or supporting structure. Here, the relevant part of the insulation barrier or supporting structure is located close to the top surface, i.e. in the environment of the hottest fluid. In alternative or complementary embodiment, the sample of a′) may be obtained from a piece of pressboard material immersed in the fluid at the top surface for the purpose of providing such samples. A benefit of the former embodiment is that no extra arrangements for the provision of the pressboard sample need to be provided. A benefit of the latter embodiment is that the pressboard sample may be accessed easily without affecting functional parts of the transformer.

In the latter case, the sample of a′) may be arranged in a fluid-permeable case immersed in the fluid at the top surface. Consequently, samples of the piece of pressboard material may be accessible from the exterior of the transformer.

In an embodiment of the first aspect, a) and b) as well as a′) and b′) are performed. This is further discussed above (the combination of the direct and the indirect approach). In such an embodiment, a) and b) may be performed more frequently than a′) and b′). For example, a) and b) may be performed more frequently than once a week and a′) and b′) may be performed less frequently than once a month.

In one embodiment of the first aspect, a) and b) are employed for updating predictions based on a′) and b′). Consequently, a) and b) may be employed to continuously update a prediction based on a′) and b′) until a new prediction based on a′) and b′) is performed. Here, the update is thus a function of the measured temperature(s) and the corresponding registered time(s) from step a) and the previous assessment from step b′). The skilled person understands that the registered times used in such a function may be the time passed since step a′) was performed (i.e. since the sample was obtained from the transformer). Since a prediction based on a′) and b′) normally require labour while a prediction based on a) and b) do not, this embodiment may provide for updated predictions at a low cost.

As a second aspect of the present invention, there is provided a transformer designed to operate at temperatures above IEC 60° C./65° C./78° C. or IEEE 65° C./65° C./80° C. comprising a tank filled with a fluid in which a core, a winding conductor covered by high temperature insulation material and pressboard material arranged as a insulation barrier and/or a supporting structure are immersed, wherein a temperature sensor and/or a fluid-permeable case for holding a piece of pressboard accessible from the exterior of the transformer is/are immersed in the fluid at its top surface.

The definitions, explanations, embodiments and benefits discussed above in connection with the first aspect apply to the second aspect mutatis mutandis. Thus, the fluid of the second aspect may for example be a high fire point fluid.

In an embodiment, the transformer of the second aspect further comprises an oxygen sensor for measuring the level of oxygen in the fluid and/or a moisture sensor for measuring the level of water in the fluid.

The fluid-permeable case of the second aspect facilitates the “direct approach” discussed above, while the temperature sensor facilitates the “indirect approach” discussed above.

The fluid-permeable case may for example comprise a removable cover which may be opened from the exterior of the transformer. Consequently, the piece of pressboard may be accessible for an operator in a convenient manner.

### Exemplary Embodiments

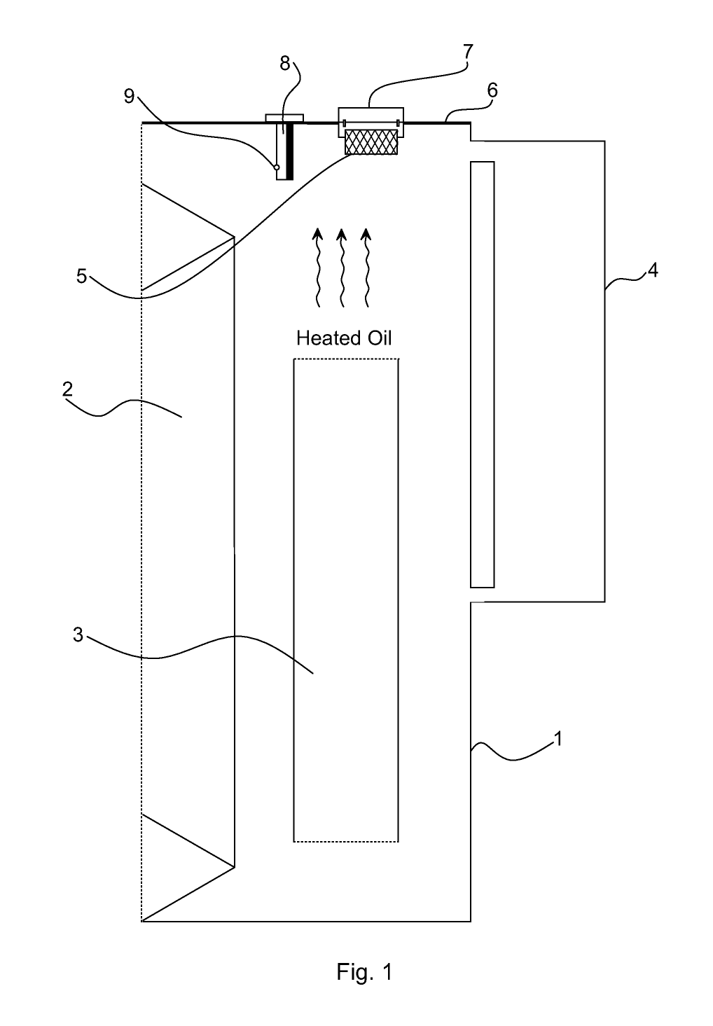

With reference to FIG. 1, there is provided an exemplary transformer, wherein a tank wall 1 and a tank bottom defines a tank. The tank is filled with a high fire point fluid, such as an ester-comprising oil. A core 2 is immersed in the fluid. The core 2 is surrounded by windings 3 in a conventional manner. The windings 3 are covered by solid high temperature-insulating material, and in the tank, pressboard material is arranged as supporting structures (not shown). During operation of the transformer, the fluid is heated (“heated oil”) and moves upwardly towards the top surface of the fluid. A cooler or radiator 4 is arranged in the tank for cooling the fluid. At the top surface of the fluid, a case 5 to hold pressboard material may be arranged in the tank cover 6. The case 5 comprises a removable cover 7, such that samples of the pressboard material may be accessed from tank exterior. Consequently, a transformer operator may obtain pressboard samples without having to open up the transformer or affecting the supporting pressboard structures. As an alternative or complement to the pressboard-containing case 5, a fluid temperature sensor 8 may be arranged in the tank such that the temperature of the fluid at the top surface may be measured. For example, a conventional oil pocket 9, may be arranged in the tank cover 6 for holding the fluid temperature sensor 8 and facilitating the temperature measurements.

As mentioned above, the solid high-temperature insulating material covers the winding conductors of the transformer of the present invention and cellulose pressboard material is used as an insulation barrier, for spacing and in supporting structures.

The insulating and pressboard material may for example be arranged as in FIG. 4, showing part of a transformer according to the present disclosure. A core 20 is surrounded by low voltage 14 and high voltage 15 winding arrangements. The winding arrangements comprises a conductor 21 (e.g. a wire) covered by solid high-temperature conductor insulation 10. Radial 11 and axial 13 spacers of solid high-temperature insulation material are arranged outside the conductor insulation 10. Static rings 12 of insulation material are arranged on the top of the winding arrangements 14, 15. If necessary, the static rings 12 may have high temperature insulation. Angle rings 16 and insulation barriers 17 of pressboard material are arranged outside and between the winding arrangements. Radial spacers (or blocks) 18 of pressboard material are arranged on the top of and above the static rings 12. A pressing structure 19 is arranged on the top of block 18 such that its lower surface is in contact with the upper surface of the highest located blocks 18. Consequently, the pressboard components of the transformer function as insulation barriers and supporting structures (pressboard components of the transformer keep the winding arrangements 14, 15 in position).

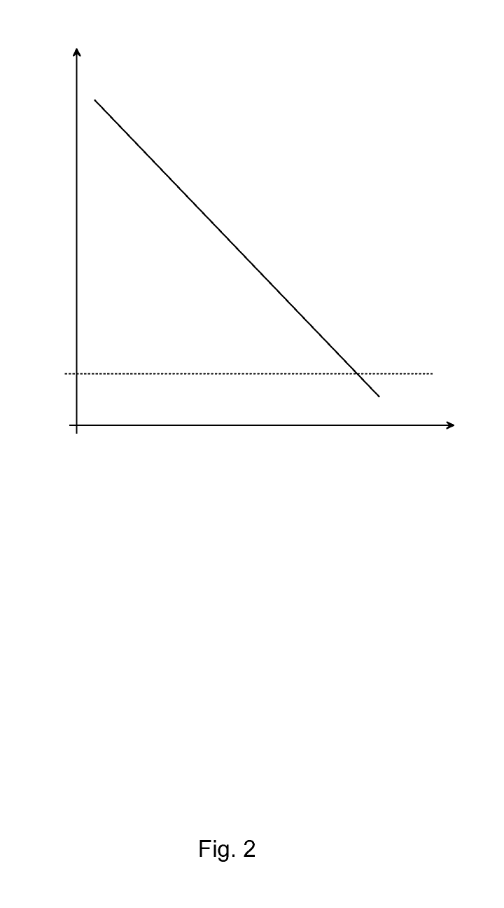

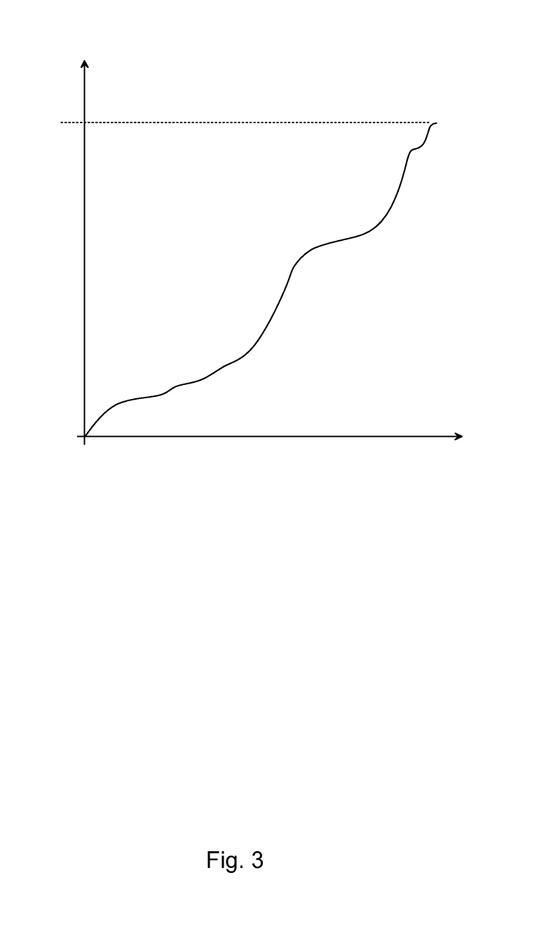

With reference to FIGS. 2 and 3, a remaining life time prediction based on measurements of the top oil temperature in the transformer is described. As explained above, this embodiment of the invention is based on that the deterioration rate of the pressboard material (which is the weakest point of this type of transformer) depends on the temperature of the fluid in which it is immersed and the time exposed to those temperatures. Higher temperatures result in higher deterioration rates. Further, the measurements can be extended to also include the level of oxygen and/or water in the fluid. Such an empirical model of the deterioration at a certain temperature may be obtained by measuring an aging variable of pressboard material that has been in contact with fluid at the temperature in question (the aging variable may for example be one of the chemical or mechanical properties of the pressboard material mentioned above). An example of such an empirical model is shown in FIG. 2. As can be seen in the figure, after an amount of time the aging variable reaches a level which is considered to be an end-of-life threshold. Preferably, several aging curves as the one in FIG. 2 representing different temperatures are obtained. An aging curve representing a higher temperature reaches the end-of-life threshold earlier, and an aging curve representing a lower temperature reaches the end-of-life threshold later. Aging curves for various oxygen and/or water levels may also be obtained in a similar fashion.

A life consumption model may then be based on the aging curves, and an example of such a model is shown in FIG. 3. The remaining life of the transformer is consumed at different rates at different points of time depending on the temperature. That is, during a period of a lower temperature, the remaining life is consumed at a lower rate than during a period of higher temperature. Eventually, the transformer reaches a point where it should be replaced. This is represented by the dotted line in FIG. 3.

Using the teachings of the present disclosure and common general knowledge, the person of skill in the art would be able to formulate a calculation algorithm by means of which the remaining lifetime of a transformer can be assessed if information about the top oil temperature and the corresponding time is obtained.

A remaining lifetime can be assessed as a continuous calculation of the integral of a temperature and time dependant function over time. For example, temperature measurements may be performed regularly, and the assessment of the remaining lifetime may be updated after each temperature measurement. The temperature measurements may for example be performed once a minute, once an hour, once a day, once a week or once a month. The skilled person understands that the prediction become more accurate if temperature measurements are performed with shorter intervals. The frequency of the measurements may be adapted to the expected load variations. That is, if the load (and thereby the top oil temperature) is expected to vary heavily, it is preferred to measure more frequently, such as at least once a day.

1. A method of assessing the remaining lifetime of a transformer operating at temperatures above IEC 60° C./65° C./78° C. or IEEE 65° C./65° C./80° C. comprising a core, a winding conductor covered by high temperature insulation material and pressboard material arranged as an insulation barrier and/or a supporting structure, wherein said covered winding conductor and said pressboard material are immersed in a fluid having a fire point of at least 300° C., said method comprising the steps of:
a) at least twice measuring the temperature of the fluid at its top surface and registering the time of each measurement; and
b) assessing the remaining lifetime of the transformer as a function of the measured temperatures and the corresponding registered times,
and/or
a′) obtaining a sample of pressboard material that has been in contact with the fluid at its top surface; and
b′) analysing the sample to assess the remaining lifetime of the transformer,
wherein the high temperature insulation material is as defined in IEC 60076-14:2009 or IEEE 1276:1997.

2. The method according to claim 1, wherein the function of b) is based on at least one empirically determined aging curve of the pressboard material.

3. The method according to claim 1, wherein the sample of a′) is obtained from the insulation barrier or supporting structure.

4. The method according to claim 1, wherein the sample of a′) is obtained from a piece of pressboard material immersed in the fluid at the top surface for the purpose of providing such samples.

5. The method according to claim 4, wherein the sample of a′) is arranged in a fluid-permeable case immersed in the fluid at the top surface such that samples the piece of pressboard material are accessible from the exterior of the transformer.

6. The method according claim 1, wherein steps a) and b) are performed to provide an update of a previous assessment of the remaining lifetime of the transformer obtained according to steps a′) and b′), which update is a function of the measured temperature(s), the corresponding registered time(s) and the previous assessment.

7. The method according to claim 1, wherein the fluid substantially consists of ester based fluids.

8. The method according to claim 1, wherein the pressboard material comprise paper or cellulose.

9. The method according to claim 1, wherein the pressboard material substantially consists of paper or cellulose and optionally glue.

10. A transformer designed to operate at temperatures above IEC 60° C./65° C./78° C. or IEEE 65° C./65° C./80° C. comprising a tank filled with a fluid having a fire point of at least 300° C. in which a core, a winding conductor covered by high temperature insulation material and pressboard material arranged as a insulation barrier and/or a supporting structure are immersed,
wherein a temperature sensor and/or a fluid-permeable case for holding a piece of pressboard accessible from the exterior of the transformer is/are immersed in the fluid at its top surface.

11. The transformer according to claim 10, wherein the fluid-permeable case comprises a removable cover which may be opened from the exterior of the transformer such that the piece of pressboard is accessible.

12. The transformer according to claim 10, further comprising an oxygen sensor for measuring the level of oxygen in the fluid.

13. The transformer according to claim 10, further comprising a moisture sensor for measuring the level of water in the fluid.
